(19) United States
(12) Patent Application Publication
(10) Pub. No.: US 2018/0127826 A1
(43) Pub. Date: May 10, 2018

(54) METHOD FOR THE PROGNOSIS AND/OR DIAGNOSIS OF AN ILLNESS BASED ON A SAMPLE OF ADIPOSE TISSUE, AND A KIT FOR SAID METHOD

(71) Applicant: LIPOZYT MARKER UG, Bremen (DE)

(72) Inventors: Helge Wilhelm THIES, Bremen (DE); Markus KLEMKE, Bremen (DE)

(73) Assignee: LIPOZYT MARKER UG, Bremen (DE)

(21) Appl. No.: 15/570,573

(22) PCT Filed: Apr. 28, 2016
(86) PCT No.: PCT/EP2016/059556

§ 371 Date: Oct. 30, 2017

(30) Foreign Application Priority Data
Apr. 30, 2015 (DE) 10 2015 208 083.8

(51) Int. Cl. C12Q 1/6883

(52) U.S. Cl. CPC C12Q 1/6883; C12Q 2600/158

(57) ABSTRACT

The invention relates to a method for the prognosis of adiposity and the prognosis and/or diagnosis of a disease selected from the group consisting of diabetes, cardiovascular diseases and metabolic syndrome.

The present invention relates to a method for the prognosis of adiposity and for the prognosis and/or diagnosis of a disease selected from the group consisting of diabetes, cardiovasular diseases and metabolic syndrome based on a sample of adipose tissue, wherein grouping of the donor into one of at least four risk groups takes place. Furthermore, the invention relates to a kit comprising suitable primer pairs for the method of the invention.

The increasing spreading of overweight and obesity constitutes a growing health problem worldwide. Above all obesity is associated with cardiovascular diseases (CVD), an elevated risk for type II diabetes mellitus and/or the development of metabolic syndrome. Overweight and obesity are defined via the Body-Mass Index (BMI), the latter describes the ratio of body weight to body size. The BMI is determined according to the following formula: BMI=body weight [kg]/body size squared [m²]. According to WHO criteria, the normal BMI lies between 18.5 and 24.9 kg/m², overweight exists at a BMI of over 25 kg/m², whereas obesity is mentioned from a BMI of 30 kg/m². Obesity is divided into further grades: class I obesity (BMI=30-34.9), class II obesity (BMI=35-39.9), class III obesity (BMI≥40) and super obese (BMI≥50). However, a high BMI says little about the actual body tissue composition and is only a first rough indicator with regard to the possibility of contracting obesity-associated diseases. More recent studies show that individuals with a high BMI (BMI≥30 kg/m²) do not necessarily have to suffer from cardiovascular diseases and type II diabetes mellitus (so-called “metabolically healthy obese”), whereas individuals having a normal BMI (18.5-24.9 kg/m²) may suffer from the said diseases in spite of their low BMI.

The cause of the emergence of overweight and obesity are growth dynamics in the adipose tissue which are triggered if the energy uptake due to food exceeds the energy consumption over a longer time. Excess energy is then stored in the adipose tissue in the form of triglycerides. The increase of adipose tissue in the body, which may lead to overweight and obesity, is based both on the rise in the number of adipocytes (hyperplasia) and on the increase in the volume of the adipocytes (hypertrophy). Hypertrophy is then seen as an initial feature of the start of overweight and possible future obesity. Since the adipocytes are able only to a limited extent to grow and to store triglycerides, the regeneration of adipocytes is absolutely necessary in the case of too high energy uptake. However, the increase in the number of adipocytes may be observed both in the case of emerging or increasing overweight and in the normal formation of adipose tissue.

During adipogenesis, the pre-adipocyte, which is similar to a fibroblast, differentiates to the lipid-storing, insulin-sensitive mature adipocyte. This differentiating process is extremely dynamic and runs over several stages of development, in which diverse transcription factors participate. The starting point of adipogenesis is the mesenchymal stem cell (MSC), which is capable, due to self-renewal, of keeping the stem cell pool constant and on the other hand may differentiate into diverse tissue types after a corresponding stimulus. Tang et al. (Tang W, Zeve D, Suh J M, Bosnakovski D, Kyba M, Hammer R E, Tallquist M D, Graff J M (2008) White fat progenitor cells reside in the adipose vasculature. Science 322:583-6) could detect these precursor cells in the perivascular niche of adipose tissue. If the MSC differntiates to the committed pre-adipocytes, a phase of cell division follows up to a point at which the pre-adipocytes will halt their growth by contact inhibition and are locked in the cell cycle. After a hormonal stimulus, the growth-locked pre-adipocytes again enter the cell cycle and pass through a phase of increased proliferation which is designated as mitotic clonal expansion. After clonal expansion there follows renewed cell cycle locking of the pre-adipocytes and the terminal differentiation starts with the transcriptional activation of certain adipocyte-specific genes. A cascade of transcription factors is activated step-wise and leads to the insulin-sensitive mature adipocyte.

Hitherto, there are only imprecise and indirect markers, such as for example abdominal overweight, high blood pressure and elevated blood glucose values etc., to estimate the risk of contracting obesity or one of the following obesity-associated diseases in future: metabolic syndrome, CVD and type II diabetes mellitus. Reliable and meaningful predictive markers do not exist to this day for the said diseases. Although overweight constitutes an important risk factor for these diseases, it is generally recognized that body adipose tissue distribution and accumulation of immature adipose tissue represent an additional, independent factor. It is known that dysfunctions during adipogenesis lead to accumulation of immature fat cells, as a result of which in long-term the formation of a diabetic disease (type II diabetes mellitus) and of metabolic syndrome is favoured. To this day there is no reliable diagnosis method to identify immature adipose tissue on the molecular level.

The puncturing of subcutaneous adipose tissue (WAT) is a simple, safe and not very invasive method of obtaining adipose tissue, but the aspirates contain too few cells to guarantee reliable molecular analysis of these cells. Particularly genes weakly expressed in the WAT cannot always be detected reliably. Below, biomarkers and methods are described which facilitate the identification of individuals with immatureadipose tissue, based, inter alia, on quantifications of gene expression in samples of WAT.

The HMGA2 protein plays an important role in many biological processes, such as growth, proliferation and differentiation. Rearrangements of the chromosome region 12q13-15, which are associated with activation of otherwise inactivated HMGA2 alleles, often lead to lipomas, benign tumours of the adipose tissue cells. With regard to normal cells, studies could verify the significant influence of HMGA2 during the growth and the development of adipocytes. EP 2 682 752 A1 discloses that based on the expression level of HMGA2, a prognosis and/or diagnosis of diabetes may take place. Furthermore, this document points to the fact that the HMGA2 expression level and the PPAR-gamma expression level behave reciprocally to one another. However, research by the applicant has shown that the risk grouping pattern disclosed in this document is too unrefined.

Therefore, it was the object of the present invention, starting from the state of the art, in particular EP 2 682 752 A1, to indicate an improved prognosis method and/or diagnosis method. This object is achieved by a method for the prognosis of obesity and for the prognosis and/or diagnosis of a disease selected from the group consisting of diabetes, cardiovascular diseases and metabolic syndrome, comprising the steps:

a) providing a sample of adipose tissue from a donor,

b) determining the gene expression level of the genes for HMGA2 and PPAR-gamma in the sample and

c) grouping the donor of the sample into one of at least four risk groups taking into account the gene expression levels of the genes for HMGA2 and PPAR-gamma in the sample and the body fat percentage, in particular the BMI of the donor, wherein classification into one of the groups takes place selected from the group consisting of
a) elevated relative gene expression level for HMGA2, non-elevated relative gene expression level for PPAR-gamma and elevated body fat proportion, in particular average BMI≥25,
b) non-elevated relative gene expression level for HMGA2, non-elevated relative gene expression level for PPAR-gamma and elevated body fat proportion, in particular average BMI≥25,
c) non-elevated relative gene expression level for HMGA2, non-elevated gene expression level for PPAR-gamma and non-elevated body fat proportion, in particular average BMI<25,
d) non-elevated relative gene expression level for HMGA2, elevated relative gene expression level for PPAR-gamma and non-elevated body fat proportion, in particular average BMI<25,

The adipose tissue used preferably comprises white adipose tissue, and more preferably it consists thereof.

In the method of the invention, individuals having immature adipose tissue are identified based on gene expression analysis, that is, low levels of PPAR-gamma expression and an elevated level of HMGA2 expression point to an elevated proportion of immature fat cells in the adipose tissue. Conversely, a high PPAR-gamma expression and a low HMGA2 expression point to an elevated proportion of mature fat cells in the adipose tissue. By means of the combination of gene expression of these two genes, it is possible to make statements about the adipose tissue composition of the individual. Using this value combination, conclusions may be drawn about, inter alia, the risk of developing diabetes.

It is surprising here that on the basis of the method of the invention, not only two risk groups, as were to be expected from EP 2 682 752 A1, can be derived, but at least 4: The said published application in fact assumes that the expression levels of HMGA2 and PPAR-gamma are always found in a significant inverse correlation. This would mean that from measuring the expression level of the one marker, the possible findings, which would be obtained from measuring the expression level of the other marker, could also be obtained. Therefore the skilled person would have been dissuaded, simply for economic reasons, from measuring both markers in parallel for a corresponding method. However, it has emerged, surprisingly, that the correlation asserted in the said published application is shown differently just with regard to the risk of contracting the said diseases: There are cases which are just not correlated conversely with regard to the two markers on the expression level. Reference is made in particular also to the examples regarding this.

The term “diagnosis” means, within the scope of this text, a forecast of elevated probability of the development and the occurrence of a clinical condition or a disease.

The term “determination” of a disease means, within the scope of this text, that a disease which already shows clinical symptoms is identified.

“Diabetes” within the scope of the present text comprises in particular the forms of type I and type II diabetes mellitus in human beings and animals, where particularly preferably type II in the human being is meant.

“Cardiovascular diseases” within the scope of this present text are preferably coronary heart disease, arteriosclerosis, hypertension, myocardial infarction, peripheral arterial occlusive disease and cardiomyopathy. “Adipose tissue” within the scope of this text consists of fat cells (adipocytes, pre-adipocytes) stored in connective tissue, and immune cells, fibroblasts and blood vessels.

A “donor” within the scope of this text is a human being or an animal, preferably a human being.

Determination of the gene expression level within the scope of the present invention may take place in any manner known to the skilled person. Determination of the gene expression level is preferably on the mRNA level or on the protein level.

“Risk groups” within the scope of this text are those groups which may be separated from one another by suitable differentiating features and in each case have a common elevated or non-elevated risk with regard to the development or the presence of a disease.

“HMGA2” within the scope of this text is the High Mobility Group AT-hook protein 2 (HMGA2) or its gene or the associated mRNA and/or parts of this protein or gene (or its mRNA), preferably at least one amino acid chain 7 amino acids, further preferably ≥15 amino acids and particularly preferably ≥20 amino acids or a nucleic acid chain of ≥20 nucleic acids, further preferably ≥40 nucleic acids and particularly preferably ≥55 nucleic acids optionally per strand. HMGA2 is a transcription factor which influences regulation of gene expression and belongs to the group of High Mobility Group A Proteins (HMGA proteins). The HMGA proteins are chromatin-associated, acid-soluble non-histone proteins which bind to sequence-independent, specific motifs of DNA. As architectonic transcription factors, they increase or inhibit the binding ability of further transcription factors via structural changes in chromatin organization. The human HMGA2 gene is localized in the chromosomal region 12q14˜15 and consists of five exons which extend over a region 160 kb long. It codes for a 109 amino acids-long protein, the molecular mass of which is 12 kDa. The HMGA2 protein is characterized by three highly conserved DNA-binding domains, the so-called AT-hooks and an acidic, negatively charged, C-terminal domain.

“PPAR-gamma” within the scope of this text is the peroxisome proliferator-activated receptor gamma (PPAR-gamma), preferably PPAR-gamma isoform 2 or its gene or the associated mRNA and/or parts of this protein or gene (or its mRNA), preferably at least one amino acid chain 7 amino acids, further preferably ≥15 amino acids and particularly preferably ≥20 amino acids or a nucleic acid chain of ≥20 nucleic acids, further preferably ≥40 nucleic acids and particularly preferably ≥55 nucleic acids optionally per strand. PPAR-gamma is a ligand-binding nuclear transcription factor of the PPAR sub-family which belongs to the group of nuclear hormone receptors. PPAR-gamma activates the transcription of various genes via heterodimerization using the retinoid X receptor a (RXRa). The human PPAR-gamma gene is localized in the chromosomal band 3p25 and consists of 11 exons. The human PPAR-gamma gene codes for 3 isoforms which in each case constitute a 477, a 505 and a 186 amino acids-long protein.

Within the scope of this text, the body fat percentage is preferably determined by means of the following method: BMI (Body-Mass Index), ABSI (A Body Shape Index), WHR (Waist-to-Hip Ratio), WtHR (Waist to Height Ratio), body fat mass, subcutaneous adipose tissue mass, visceral adipose tissue mass, UWW (Under Water Weighing), ADP (Air Displacement Plethysmography), calipometry, DEXA (Dual Energy X-ray Absorptiometry), BIA (Bioelectrical Impedance Analysis), BIVA (Bioelectrical Impedance Vector Analysis) and MRI (Magnetic Resonance Imaging), where the BMI is preferred.

It is preferred within the scope of the present invention that the donor is a human being and/or the disease is type II diabetes mellitus.

The data available to the inventors and the experimental experience have shown that the method of the invention is suitable in particular for the prognosis and/or diagnosis of the disease type II diabetes mellitus.

It is preferred for the method of the invention if the sample has been obtained by puncturing the subcutaneous abdominal adipose tissue. Cells and cell clusters, which facilitate molecular-genetic analysis, can be obtained particularly well by fan-like puncturing with suction. The fan-like procedure during puncture firstly reduces adhesion/clogging of the cannula tip with fat cells, secondly, cells are obtained from various regions of the relevant adipose tissue and thus there is a representative cross-section of distribution of different adipose tissue cell types.

A method of the invention is particularly preferred wherein the sample mass is ≤50 mg, preferably ≤20 mg and particularly preferably ≤5 mg.

Surprisingly, it has been shown that even for very small sample volumes, reliably differentiated results may be achieved. It is here particularly preferred if the sample has been obtained by puncturing as a fine-needle aspirate, as described in Example 1 under fine-needle aspiration.

According to the invention, the method is preferably carried out so that determination of the gene expression level takes place on the mRNA level.

There are a host of techniques for determination on the mRNA level. Nevertheless, it is surprising that on the mRNA level, even for very small sample quantities, reliable and differentiated results are achieved using the method of the invention.

For determination of gene expression levels on the mRNA level, usually the steps of RNA isolation or at least accumulation, subsequent cDNA synthesis and then a quantitative PCR are usual.

All methods familiar to the skilled person may be used for RNA isolation, just as for cDNA synthesis.

Preferred methods for quantification of gene expression are array hybridization (DNA or protein arrays), mass spectrometry (MALDI-TOF), serial analysis of gene expression (SAGE) and other methods for the detection of mRNAs or proteins or fragments thereof; in particular here, quantitative real-time PCR (qRT PCR) is preferred.

However, in principle all methods are possible which lead to a reliable gene expression measurement.

A method of the invention is preferred wherein before execution of the real-time PCR, pre-amplification of the cDNA takes place.

Even more precise results can thus be obtained which in addition are also reproducible.

It is particularly preferable for the method of the invention if the grouping in step c) takes place using the multivariate model of the self-organizing maps according to Kohonen.

Reference is also made to Example 3 for using the multivariate model of the self-organized maps according to Kohonen. Surprisingly, it has been shown that with classification by means of the said statistical method, four donor groups are developed therefrom very clearly. This was not to have been expected according to the state of the art. Reference is made regarding this to the statements made above.

A method of the invention is preferred wherein classification into one of the groups takes place selected from the group consisting of

a) elevated relative gene expression level for HMGA2, non-elevated relative gene expression level for PPAR-gamma and elevated body fat proportion, in particular average BMI≥25, preferably ≥28, further preferably ≥30,

b) non-elevated relative gene expression level for HMGA2, non-elevated relative gene expression level for PPAR-gamma and elevated body fat proportion, in particular average BMI≥25, preferably ≥28, further preferably ≥30,

c) non-elevated relative gene expression level for HMGA2, non-elevated gene expression level for PPAR-gamma and non-elevated body fat proportion, in particular average BMI<25 and preferably <23,

d) non-elevated relative gene expression level for HMGA2, elevated relative gene expression level for PPAR-gamma and non-elevated body fat proportion, in particular average BMI<25, preferably <23,

e) elevated relative gene expression level for HMGA2, non-elevated gene expression level for PPAR-gamma and non-elevated body fat proportion, in particular average BMI<25 and preferably <23,

f) non-elevated relative gene expression level for HMGA2, elevated relative gene expression level for PPAR-gamma and elevated body fat proportion, in particular average BMI≥25, preferably ≥28, further preferably ≥30. These six groups, relative to the statements possible hitherto in the prior art, additionally enable the following information to be obtained:

group a) is exposed to an elevated risk of developing, inter alia, sarcopenia, hyperinsulinemia, hyperglycemia, insulin resistance, type II diabetes mellitus, lipometabolic disturbances, inflammations, cardiovascular diseases and cancer or even of being affected thereby undiagnosed.

group b) is exposed to a normal or slightly elevated risk of developing, inter alia, hyperinsulinemia, hyperglycemia, insulin resistance, type II diabetes mellitus, and a low risk of cardiovascular diseases or even of being affected thereby undiagnosed.

group c) is exposed to an elevated risk of developing, inter alia, sarcopenia, chronic diseases, insulin resistance, type II diabetes mellitus, obesity, lipometabolic disturbances, inflammations, cardiovascular diseases and cancer or even of being affected thereby undiagnosed.

group d) is exposed to a low risk of developing, inter alia, sarcopenia, hyperinsulinemia, hyperglycemia, insulin resistance, type II diabetes mellitus, obesity, lipometabolic disturbances, inflammations and cardiovascular diseases or even of being affected thereby undiagnosed.

group e) is exposed to an elevated risk of contracting, inter alia, sarcopenia, hyperinsulinemia, hyperglycemia, insulin resistance, type II diabetes mellitus, lipometabolic disturbances, inflammations, cardiovascular diseases and cancer or even of being affected thereby undiagnosed.

group f) is exposed to a low risk of contracting, inter alia, sarcopenia, hyperinsulinemia, hyperglycemia, insulin resistance, type II diabetes mellitus, adiposity, lipometabolic disturbances, inflammations and cardiovascular diseases or even of being affected thereby undiagnosed. In particular groups c) and e) thus constitute two risk group not stipulated in the prior art.

“Elevated relative gene expression level” in this context means that the gene expression level is elevated with respect to the mean of the gene expression levels of a group of patients (preferably n greater than 100). Within the framework of the relative quantification, the expression of the target genes is ascertained in relation to a so-called endogenous control. Ubiquitously expressed housekeeping genes serve as an endogenous control, preferably selected from the group consisting of HPRT, 18S, GAPDH, GUSB, PBGD, B2M, ABL, RPLP0, wherein most particularly HPRT is preferred.

Such a gene expression level value preferably counts as elevated if it deviates from the respective reference value at the top by at least 5%, further preferably at least 10%, particularly preferably at least 20%.

A non-elevated gene expression level within the scope of the present application is in contrast one such that moves in the order of magnitude of one or more of the aforementioned (selected) reference parameters (that is, +/−2% from the corresponding value) or—which is preferred—one such which is reduced with respect to one or more of the reference values, i.e., is reduced by at least 5%, preferably at least 10% and further preferably at least 20%. Part of the invention is also a kit for a method of the invention, comprising

a) a primer pair which binds to the cDNA (primer may also bind to DNA) of HMGA2 and

b) a primer pair which binds to the cDNA (primer may also bind to DNA) of PPAR-gamma.

A primer which binds to a certain cDNA here is one such which can bind to the target cDNA due to its nucleotide sequence under stringent conditions for hybridization.

Stringent conditions are here defined as equivalent to hybridization in 6×SSC at 45° C. followed by a washing step in 0.2×SSC, 0.1% SDS at 65° C. (Current Protocols in Molecular Biology, John Wiley & Sons, N.Y., 6.3.1-6.3.6, 1991).

A primer pair here are 2 primers which bind to sections of the respective cDNA on different strands under stringent conditions.

The kit of the invention may preferably contain one or more further of the components listed below:

Fluorescence-labeled DNA probes, reverse transcriptases, polymerases, hexanucleotides, nucleotides, RNase inhibitors, syringes and cannulae. In the following examples, overweight is defined as a BMI>25.

Adipose tissue samples were obtained either by means of fine-needle aspiration or during surgery. After RNA isolation, cDNA synthesis and cDNA pre-amplification, expression of the mRNA of HMGA2 and PPAR-gamma was determined by means of quantitative real-time PCR.

If mean is mentioned in the examples, if in doubt the arithmetic mean is meant.

### EXAMPLE 1

Proof of Expression of PPAR-Gamma and HMGA2 in Adipose Tissue Punctates, Obtained by Fine-Needle Aspiration of the Subcutaneous Abdominal Adipose Tissue

Material and Methods

Fine-Needle Aspiration

Fine-needle aspirates were obtained by puncturing subcutaneous abdominal hWAT by means of 20 ml syringe and a single-use injection cannula (diameter 0.90×40 mm). After disinfection of the puncture site, the cannula was introduced into the subcutaneous adipose tissue. Negative pressure was generated using the syringe and the cannula was moved back and forth in the tissue like a fan to thus aspirate cells of the adipose tissue. Directly after puncturing, the samples were taken up in 1 ml of QIAzol lysis reagent (QIAGEN, Hilden, Germany) and the cannula was rinsed several times using the QIAzol. Then the samples were frozen at −80° C.

RNA Isolation

Isolation of the total RNA took place by means of RNeasy lipid tissue mini kit (QIAGEN, Hilden, Germany) in a QIAcube (QIAGEN, Hilden, Germany) according to manufacturer's instructions. The fine-needle aspirates (5 mg) in 1 ml of QIAzol lysis reagent were homogenized in a Tissue Lyser II (QIAGEN, Hilden, Germany) and then the homogenate was incubated at room temperature for 5 minutes. There followed the addition of 200 μl of chloroform, which was mixed with the sample by means of vigorous shaking by hand for 15 seconds. The sample was again incubated for 2 minutes at room temperature and centrifuged at 12,000×g for 15 minutes at 4° C. The upper aqueous phase was then transferred to a new 2 ml cup and the total RNA was isolated via a Qiagen RNeasy mini spin column (QIAGEN, Hilden, Germany) in a QIAcube according to manufacturer's instructions.

cDNA Synthesis

For the cDNA synthesis, ≤250 ng of RNA were reverse transcribed into cDNA by means of 200 U M-MLV of reverse transcriptase, RNase Out (Invitrogen, Darmstadt, Germany) and 150 ng of random primer (Invitrogen, Darmstadt, Germany) according to manufacturer's instructions. The RNA was denatured at 65° C. for 5 minutes and then stored on ice for at least 1 minute. After addition of the enzyme, the mixture was incubated for annealing of the random primer to the RNA of the mix for 10 minutes at 25° C. The subsequent reverse transcription was carried out at 37° C. for 50 minutes, followed by 15 minutes of inactivation of the reverse transcriptase at 70° C.

Pre-Amplification of the cDNA

5 μl of cDNA were pre-amplified by means of RealTime ready cDNA Preamp Mastermix (Roche, Mannheim, Germany) using HMGA2 and HPRT (hypoxanthine phosphoribosyl-transferase 1) specific primers according to manufacturer's instructions. The pre-amplification of the cDNA took place according to the following temperature profile: 95° C. for 1 minute followed by 14 cycles at 95° C. for 15 seconds and at 60° C. for 4 minutes.

Quantitative Real-Time PCR (qRT PCR)

The relative quantification of gene expression was carried out by means of real-time PCR on the Applied Biosystems 7300 Real-Time PCR System. Commercially available gene expression assays (Life Technologies, Carlsbad, Calif., USA) were used for quantification of the mRNA levels of HMGA2 (Assay-ID Hs00171569_m1) and PPAR-gamma (Assay-ID Hs01115513_m1). HPRT was used as an endogenous control, as described by Klemke et al. (Klemke M, Meyer A, Hashemi Nezhad M, Beige G, Bartnitzke S, Bullerdiek J (2010) Loss of let-7 binding sites resulting from truncations of the 3′ untranslated region of HMGA2 mRNA in uterine leiomyomas. Cancer Genet Cytogenet 196:119-123). All reactions were measured in triplicate. The quantification of gene expression was carried out in 96-well plates using the pre-amplified cDNA to be investigated, the respective gene-specific assay and the FastStart Universal Probe Master (Rox) (Roche, Mannheim, Germany). The temperature profile of the real-time PCR followed the manufacturer's instructions: At 95° C. denaturation of the template takes place for 10 minutes. Then amplification followed in 50 cycles starting with denaturation for 15 seconds at 95° C. and the combination of annealing/elongation for 60 seconds at 60° C. The data obtained was evaluated by means of the comparative Delta-Ct method (ΔΔCT method). [(Livak K J, Schmittgen T D (2001) Analysis of relative gene expression data using real-time quantitative PCR and the 2^{-ΔΔC(T)} method. Methods 25: 402-408)].

Results

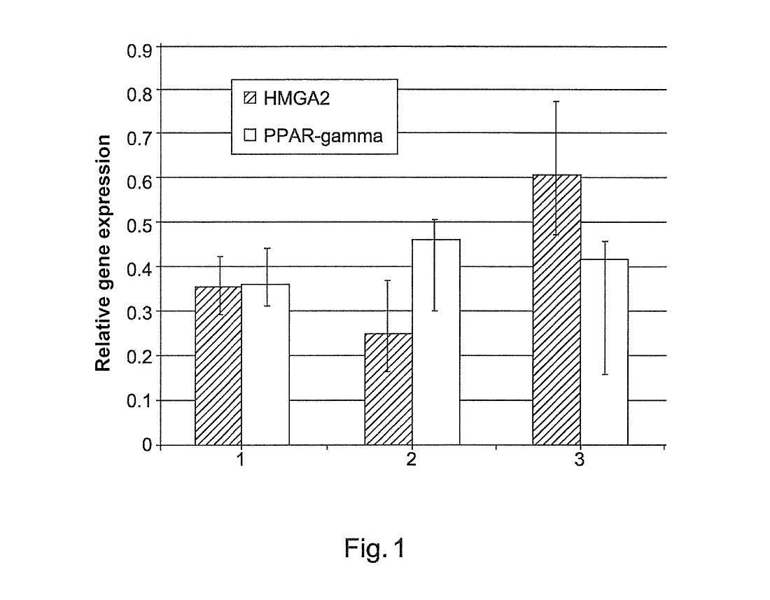

The gene expression of PPAR-gamma and HMGA2 could be measured by means of qRT PCR in three samples (FIG. 1). The results show unambiguously that HMGA2-mRNA can be quantified reliably from fine-needle aspirates using even very small quantities of adipose tissue cells. In addition, the quantity of isolated RNA from the fine-needle aspirates suffices to determine the expression of further genes after pre-amplification.

FIG. 1 shows the relative expression of HMGA2- and PPAR-gamma in three samples. It shows that reliable quantification of HMGA2 expression and PPAR-gamma expression is possible even from very small quantities of RNA (concentration≤25 ng/μl) which has been obtained by fine-needle aspiration.

### EXAMPLE 2

The Expression of HMGA2 and PPAR-Gamma Expression in Adipose Tissue Samples of Overweight Individuals

Material and Methods

Tissue Samples

The human subcutaneous abdominal adipose tissues were removed during surgery and stored in liquid nitrogen after surgery. The requirements of the Declaration of Helsinki were fulfilled for all human adipose tissue samples used. A written declaration of consent for the use of tissue samples was delivered by the patients (n=157).

RNA Isolation

Isolation of the total RNA took place by means of RNeasy lipid tissue mini kit (QIAGEN, Hilden, Germany) as described under Example 1.

cDNA Synthesis

cDNA synthesis took place likewise as described under Example 1.

Quantitative Real-Time PCR

Also the carrying out of the qRT PCR took place as described in Example 1.

Statistical Analysis

The statistical significance of the differences between the normal-weight and the overweight group was determined by means of Kendall's Tau-b test. In all comparisons, p<0.05 was regarded as statistically significant and p<0.001 as highly significant.

Results

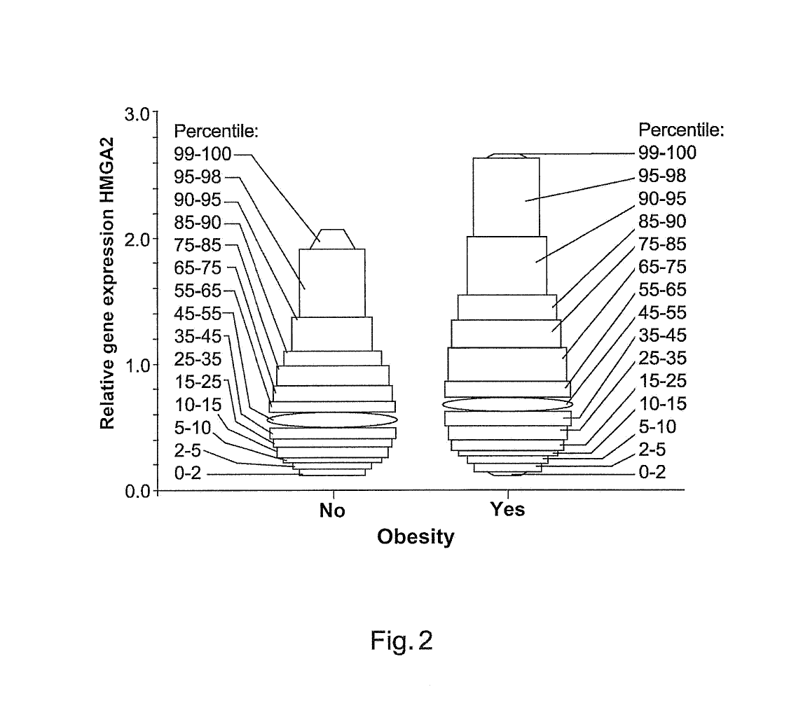

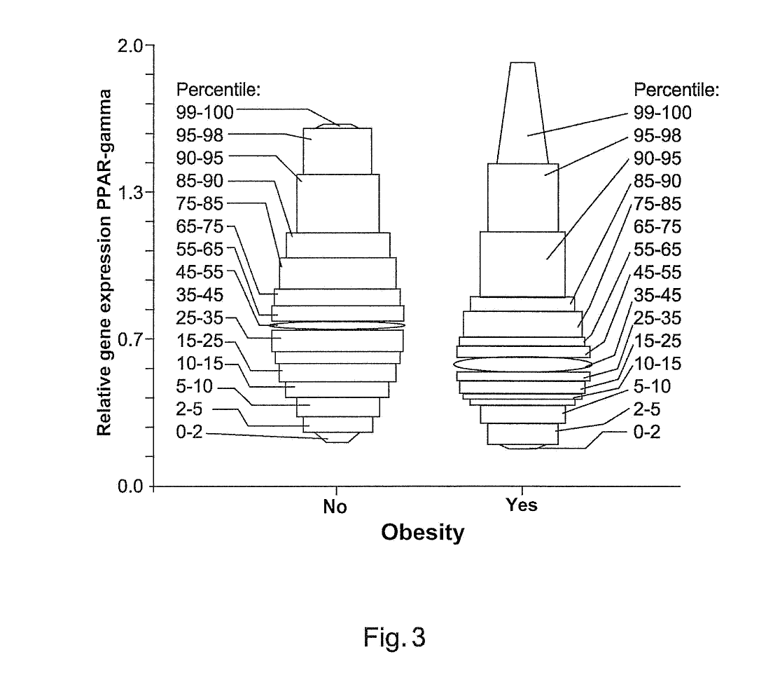

Statistically significant differences (p<0.05) in the expression of HMGA2 could be seen in samples of normal-weight individuals (n=74) compared to overweight individuals (n=83) (FIG. 2). Furthermore, highly significant differences in the PPAR-gamma-mRNA level were detected in samples of normal-weight individuals (n=74) compared to overweight individuals (n=83) (FIG. 3).

FIG. 2 shows the HMGA2 expression of normal-weight and overweight individuals as a percentile plot. The 15 percentiles are delimited from one another as follows: 0-2, 2-5, 5-10, 10-15, 15-25, 25-35, 35-45, 45-55, 55-65, 65-75, 75-85, 85-90, 90-95, 95-99, 99-100.

It can be seen from FIG. 2 that almost every percentile in the overweight group has a higher gene expression than the corresponding percentile of the respective normal-weight group.

FIG. 3 shows the PPAR-gamma expression of normal-weight and overweight individuals as a percentile plot. The 15 percentiles are delimited from one another as follows: 0-2, 2-5, 5-10, 10-15, 15-25, 25-35, 35-45, 45-55, 55-65, 65-75, 75-85, 85-90, 90-95, 95-99, 99-100.

FIG. 3 shows that almost every percentile in the normal-weight group has a higher gene expression than the corresponding percentile of the respective overweight group.

### EXAMPLE 3

Data Analysis by Means of Self-Organizing Maps Shows Four Different PPAR-Gamma and HMGA2 Gene Expression Profiles

Material and Methods

Tissue Samples

The human subcutaneous abdominal adipose tissues were removed during surgery and stored in liquid nitrogen after surgery. The requirements of the Declaration of Helsinki were fulfilled for all human adipose tissue samples used. A written declaration of consent for the use of the tissue samples was given by the patients (n=157).

Further Steps

RNA isolation, cDNA synthesis and qRT PCR took place as described in Example 2.

Statistical Analysis

In order to analyse the relationship of biomarkers HMGA2 and PPAR-gamma to the BMI, Kohonen's multivariate model of self-organizing maps (see Kohonen T (2001) Self-Organizing Maps. Third, extended edition. Springer Berlin, Heidelberg, New York) was used. The aim of this analysis was to divide the test subjects/patients into groups (segments) so that the variance of the values of BMI, HMGA2 expression and PPAR-gamma expression is minimal within the segments and is maximal between the segments.

Results

After analysis of the relationship of biomarkers HMGA2, PPAR-gamma and the BMI status by means of Kohonen's Self-Organizing Maps (SOM), surprisingly, division of the group of patients into four groups was shown and not as expected into two groups, that is, into a normal-weight and an overweight group.

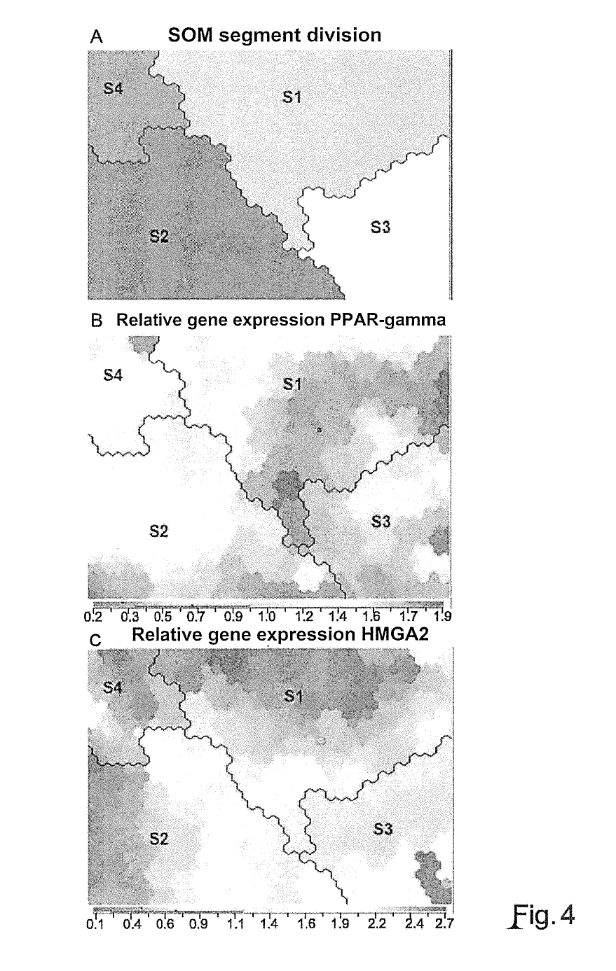

FIG. 4A shows the division of the group of patients by means of SOM according to Kohonen taking into account the relative HMGA2 expression and PPAR-gamma expression and BMI status. Two groups (S1 and S3) have a BMI average value >30 and have pronounced differences in the expression profiles of HMGA2 and PPAR-gamma. The patients having a BMI average value of <25 were likewise divided into two groups and likewise showed in each case a separate gene expression profile of HMGA2 and PPAR-gamma. Hence it could be shown that the inverse correlation shown in EP 2 682 752 A1 of HMGA2 and PPAR-gamma is only partly correct. In particular a very much more precise division into potential risk groups is possible using the methods described here based on the gene expression profiles of the patients. Mainly patients with overweight are found in segments S1 and S3, whereas patients with normal weight are found in segments S2 and S4.

FIG. 4B shows the division of the group of patients by means of SOM according to Kohonen taking into account the BMI status with reference to the relative PPAR-gamma expression (low expression=light; high expression=dark).

FIG. 4C shows the division of the group of patients by means of SOM according to Kohonen taking into account the BMI status with reference to the relative HMGA2 expression (low expression=light; high expression=dark).

The self-organizing map for PPAR-gamma (FIG. 4B) shows an elevated PPAR-gamma expression in normal-weight patients in segment S4. In contrast thereto, overweight patients in segment S3 show a high HMGA2 expression (FIG. 4C).

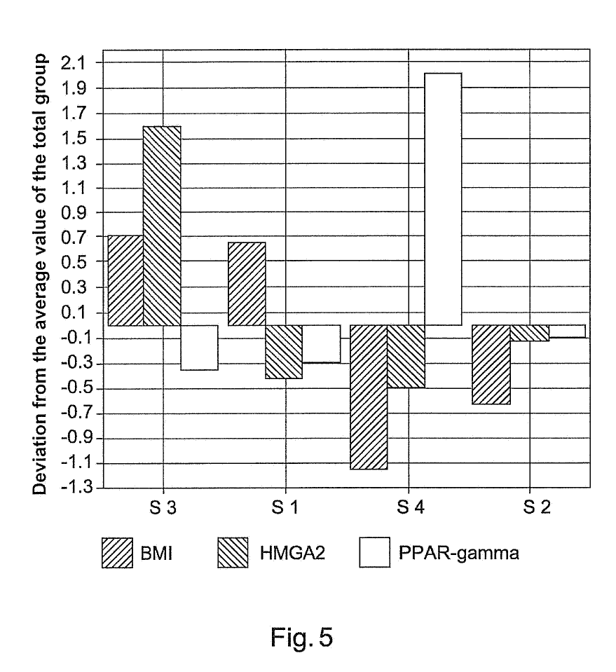

FIG. 5: shows a bar diagram of the attributes BMI, HMGA2 expression and PPAR-gamma expression in the four segments. The height of a bar in the bar diagram is the deviation of the mean of the attribute value within the segment from the respective mean of the total data record. The unit corresponds to the standard deviation from the total data record.

FIG. 5 shows the deviations of the mean of the respective attributes of a segment from the mean of the respective attribute of the total data record.

In FIG. 5 segment S4 is characterized by an elevated expression of PPAR-gamma, whereas segments S1, S2 and S3 have a low expression of PPAR-gamma. Segment S3 shows a high HMGA2 expression in contrast to segments S1, S2 and S4 which have a low HMGA2 expression.

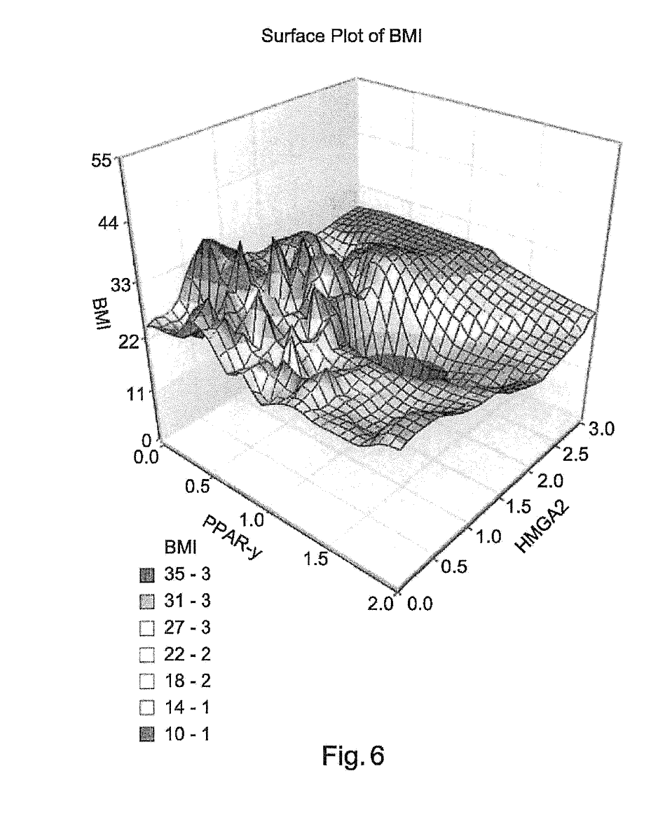

FIG. 6: Surface plot produced from the data for BMI, HMGA2 expression and PPAR-gamma expression in human subcutaneous abdominal white adipose tissue (n=157). Low PPAR-gamma expression (≤0.671) and high HMGA2 expression (≥0.717) correlate with a high BMI and vice versa.

### EXAMPLE 4

Influence of Weight Changes on the Gene Expression Profiles of HMGA2 and PPAR-Gamma

Material and Methods

Sample Preparation

Samples were obtained by means of fine-needle aspiration as described in Example 1. Also the subsequent steps of sample preparation, that is, RNA isolation, cDNA synthesis, pre-amplification of cDNA and quantitative real-time PCR took place as described in Example 1.

Results

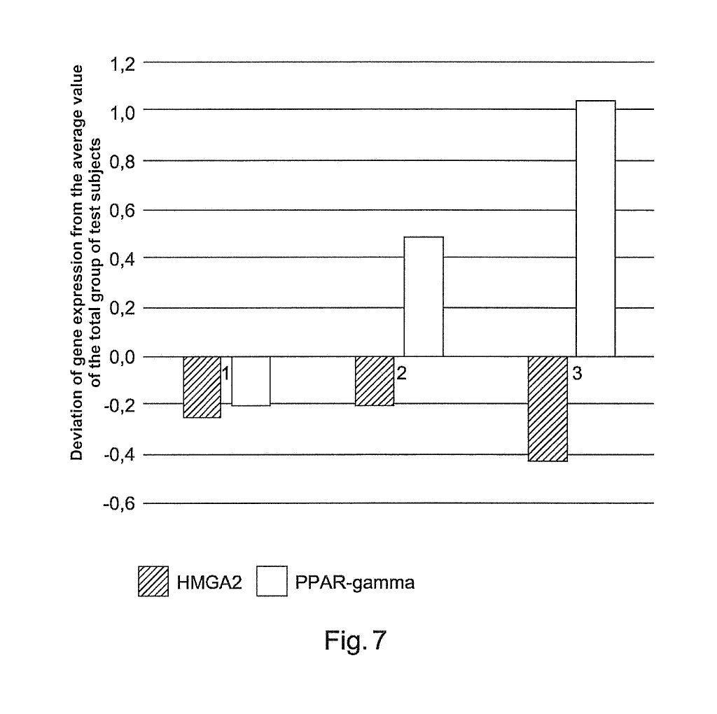

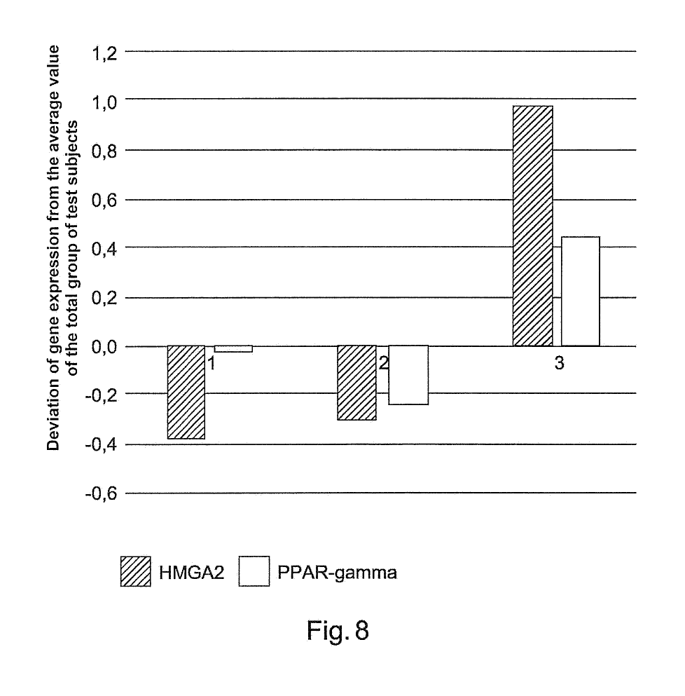

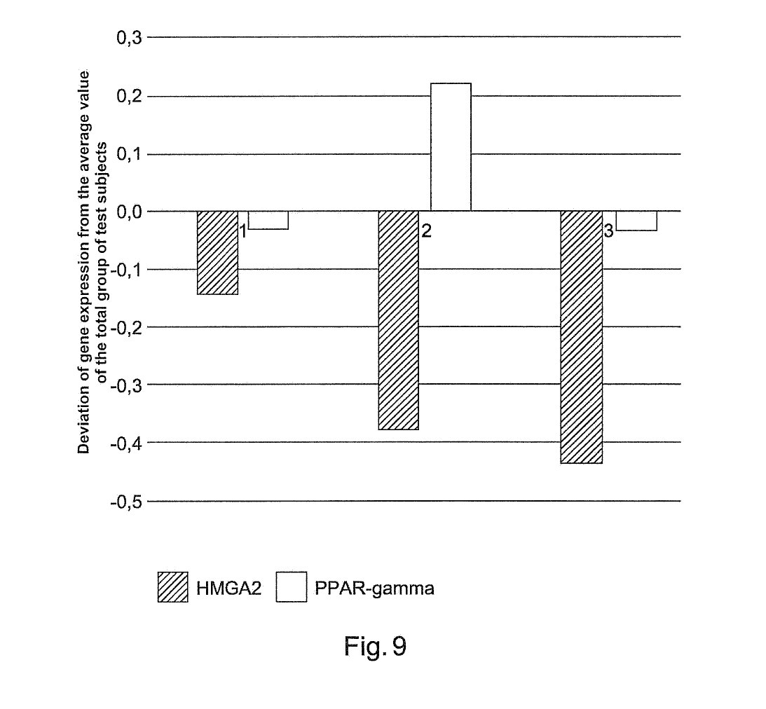

A decrease in body weight and BMI of test subjects 1 and 2 over a period of 3 months leads to changes in the gene expression profile of HMGA2 and PPAR-gamma (FIGS. 7 and 8). Test subject 1 (FIG. 7) reduced from the 1st to the 3rd measuring time his body weight by −2.7 kg and his BMI from 23.4 to 22.5, and at the same time his deviation in gene expression from the mean of the total group of test subjects of HMGA2 and PPAR-gamma changed from −0.253 and −0.207 (1st measuring time) to −0.427 or 1.039 (3rd measuring time). From the 2nd measuring point to the 3rd measuring point, the body fat percentage additionally changed by −0.9%. Test subject 2 (FIG. 8) reduced from the 1st to the 3rd measuring time his body weight by −1.8 kg and his BMI from 31.2 to 30.5, and at the same time his deviation in gene expression from the mean of the total group of test subjects of HMGA2 and PPAR-gamma changed from −0.381 and −0.033 (1st measuring time) to 0.971 and 0.440 (3rd measuring time). From the 2nd measuring point to the 3rd measuring point, the body fat percentage additionally changed by +1.8%. For test subject 3 (FIG. 9) from the 1st to the 3rd measuring time, the body weight changed by +0.2 kg and his BMI from 41.5 to 41.6, and at the same time his deviation in gene expression from the mean of the total group of test subjects of HMGA2 and PPAR-gamma changed from −0.145 and −0.034 (1st measuring time) to −0.437 and −0.036 (3rd measuring time). From the 2nd measuring point to the 3rd measuring point, the body fat percentage additionally changed by −1.3%.

These results show that the adipose tissue composition of mature and immature fat cells can be influenced by changing the body weight. However, these effects cannot be determined simply by measurements of body weight, BMI or body fat percentage, but instead require, inter alia, analysis of the gene expression profiles in order to determine change in adipose tissue composition.

FIG. 7 is the representation of deviation in gene expression of HMGA2 and PPAR-gamma of test subject 1 from the respective mean of gene expression of HMGA2 and PPAR-gamma of the total group of test subjects (n=157) at the three measuring times (total measuring period 3 months, interval between the individual measuring times in each case one month). The deviation in gene expression of HMGA2 at the following measuring times was: 1st −0.253; 2nd −0.207; 3rd −0.427. The deviation in gene expression of PPAR-gamma at the following measuring times was: 1st −0.207; 2nd 0.492; 3rd 1.039.

FIG. 8 is the representation of deviation in gene expression of HMGA2 and PPAR-gamma of test subject 2 from the respective mean of gene expression of HMGA2 and PPAR-gamma of the total group of test subjects (n=157) at the three measuring times (total measuring period 3 months, interval between the individual measuring times approximately in each case one month). The deviation in gene expression of HMGA2 at the following measuring times was: 1st −0.381; 2nd −0.297; 3rd 0.971. The deviation in gene expression of PPAR-gamma at the following measuring times was: 1st −0.033; 2nd −0.245; 3rd 0.440.

FIG. 9 is the representation of deviation in gene expression of HMGA2 and PPAR-gamma of test subject 3 from the respective mean of gene expression of HMGA2 and PPAR-gamma of the total group of test subjects (n=157) at the three measuring times (total measuring period 3 months, interval between the individual measuring times approximately in each case one month). The deviation in gene expression of HMGA2 at the following measuring times was: 1st −0.145; 2nd −0.381; 3rd −0.437. The deviation in gene expression of PPAR-gamma at the following measuring times was: 1st −0.034; 2nd 0.221; 3rd −0.036.

### EXAMPLE 5

Expression Profiles of PPAR-Gamma and HMGA2 in the Adipose Tissue of Diabetics

Material and Methods

Tissue Samples

The human subcutaneous abdominal adipose tissues were taken during surgery and stored in liquid nitrogen after surgery. The requirements of the Declaration of Helsinki were fulfilled for all human adipose tissue samples used. A written declaration of consent for the use of the tissue samples was given by the patients.

RNA isolation, cDNA synthesis and quantitative real-time PCR were carried out as described under Example 1.

Results

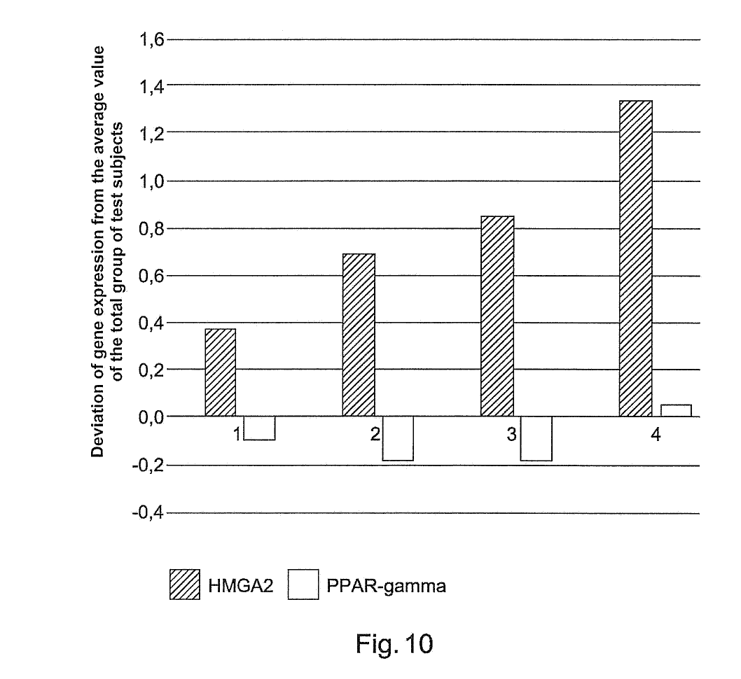

FIG. 10 shows a column diagram of the gene expression profiles of PPAR-gamma and HMGA2, produced from the deviation of mean of expression of the corresponding gene from the respective mean of gene expression of the total group of test subjects of 4 diabetics having type II diabetes mellitus. The deviation in gene expression of HMGA2 of the respective diabetic from the mean of the total group of test subjects was: 1: 0.375; 2: 0.692; 3: 0.847; 4: 1.344. The deviation in gene expression of PPAR-gamma of the respective diabetic from the mean of the total group of test subjects was: 1: −0.105; 2: −0.197; 3: −0.185; 4: 0.049.

The gene expression profiles of PPAR-gamma and HMGA2 of four type II diabetics, produced from the deviation of the mean of expression of the corresponding gene from the respective mean of expression of the total group of test subjects, are characteristic (FIG. 10). The gene expression profiles shown have a positive deviation in HMGA2 expression and a negative or extremely low PPAR-gamma expression from the mean of the total group of test subjects. These gene expression profiles point to a high proportion of pre-adipocytes and a low proportion of mature insulin-sensitive adipocytes. The BMI status of the diabetics ranged from normal-weight (diabetic 1: BMI 23.4 kg/m²) via overweight (diabetic 2: BMI 26.5 kg/m²) to class I obesity (diabetic 3: BMI 31.5 kg/m²) and class II obesity (diabetic 4: BMI 36.0 kg/m²).

These results show that the BMI as a marker for existing or developing type II diabetes mellitus disease is only a weak indicator. In contrast thereto, the gene expression profile analysis of PPAR-gamma and HMGA2 shows a characteristic profile in individuals who have contracted type II diabetes mellitus.

1. Method for the prognosis of adiposity and/or the prognosis and/or diagnosis of a disease selected from the group consisting of diabetes, cardiovascular diseases and metabolic syndrome, comprising the steps:
a) providing a sample of adipose tissue from a donor,
b) determining the gene expression level of the genes for HMGA2 and PPAR-gamma in the sample and
c) grouping the donor of the sample into one of at least four risk groups taking into account the gene expression levels of the genes for HMGA2 and PPAR-gamma in the sample and the body fat percentage, in particular the BMI of the donor, wherein classification into one of the groups takes place selected from the group consisting of
a) elevated relative gene expression level for HMGA2, non-elevated relative gene expression level for PPAR-gamma and elevated body fat proportion, in particular average BMI≥25,
b) non-elevated relative gene expression level for HMGA2, non-elevated relative gene expression level for PPAR-gamma and elevated body fat proportion, in particular average BMI≥25,
c) non-elevated relative gene expression level for HMGA2, non-elevated gene expression level for PPAR-gamma and non-elevated body fat proportion, in particular average BMI<25,
d) non-elevated relative gene expression level for HMGA2, elevated relative gene expression level for PPAR-gamma and non-elevated body fat proportion, in particular average BMI<25,

2. Method according to claim 1, wherein the donor is a human being.

3. Method according to claim 1, wherein the disease is type II diabetes mellitus.

4. Method according to claim 1, wherein the sample has been obtained by puncturing the subcutaneous abdominal adipose tissue.

5. Method according to claim 1, wherein the sample mass is ≤50 mg, preferably ≤20 mg and particularly preferably ≤5 mg.

6. Method according to claim 1, wherein determination of the gene expression level takes place on the mRNA level.

7. Method according to claim 6, wherein pre-amplification of the cDNA takes place before determination of the gene expression level.

8. Method according to claim 1, wherein the grouping in step c) takes place using the multivariate model of the self-organizing maps according to Kohonen.

9. Method according to claim 1, wherein the grouping into one of at least five or at least six risk groups takes place taking into account the groups
e) elevated relative gene expression level for HMGA2, non-elevated relative gene expression level for PPAR-gamma and non-elevated body fat proportion, in particular average BMI<25, and/or
f) non-elevated relative gene expression level for HMGA2, elevated relative gene expression level for PPAR-gamma and elevated body fat proportion, in particular average BMI≥25.

10. Method according to claim 1, wherein the gene expression level is measured on the mRNA level relative to the gene expression level of a housekeeping gene.

11. Kit for a method according to claim 1, comprising
a) a primer pair which binds to the cDNA of HMGA2 and
b) a primer pair which binds to the cDNA of PPAR-gamma
